(19) United States
(12) Patent Application Publication
(10) Pub. No.: US 2017/0204330 A1
(43) Pub. Date: Jul. 20, 2017

(54) LIQUID CRYSTAL COMPOSITION AND LIQUID CRYSTAL DISPLAY DEVICE INCLUDING THE SAME

(71) Applicant: Samsung Display Co. Ltd., Yongin-si (KR)

(72) Inventors: Seul Gee LEE, Seoul (KR); Ji Hong BAE, Yongin-si (KR); Keun Chan OH, Hwaseong-si (KR)

(21) Appl. No.: 15/215,689

(22) Filed: Jul. 21, 2016

(30) Foreign Application Priority Data
Jan. 14, 2016 (KR) 10-2016-0004897
Apr. 12, 2016 (KR) 10-2016-0044665

(51) Int. Cl. C09K 19/30; G02F 1/1343; C07C 43/225; G02F 1/1339; C07C 13/28; C07C 43/21; G02F 1/1368; G02F 1/1337

(52) U.S. Cl. CPC C09K 19/3098; G02F 1/1368; G02F 1/134309; G02F 1/133711; G02F 1/13394; C07C 13/28; C07C 43/21; C07C 43/225; G02F 2001/133726; G02F 2001/13398; C07C 2101/14; C07C 2101/16

(57) ABSTRACT

A liquid crystal composition includes: at least one of compounds represented by Formula 1:

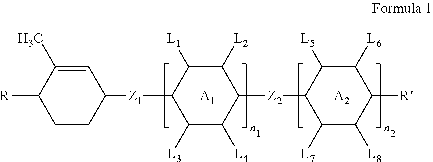

in the Formula 1, R—* and R′—* are each independently any one of *—H, *—F, *—Cl, *—I, *—Br, a C₁₋₁₂ alkyl group, a C₁₋₁₂ alkoxy group, and a cyano group; *—Z₁—* and *—Z₂—* are each independently any one of *—COO—*, *—OCO—*, *—CF₂O—*, *—OCF₂—*, *—CH₂O—*, *—OCH₂—* *—SCH₂—*, *—CH₂S—*, *—C₂F₄—*, *—CH₂CF₂—*, *—CF₂CH₂—*, *—(CH₂)ₘ—*, *—CH═CH—*, *—CF═CF—*, *—CH═CF—*, *—CF═CH—*, *—C≡C—*, *—CH═CHCH₂O—*, or a single bond;

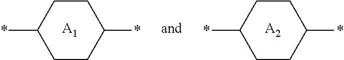

and are each independently any one of

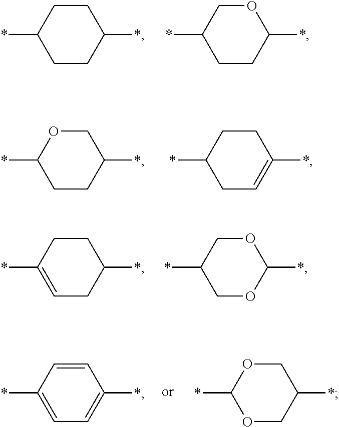

n₁ and n₂ are each independently 0 to 3; m is 2 to 5; and L₁-*, L₂-*, L₃-*, L₄-*, L₅-*, L₆-*, L₇-*, and L₈-* are each independently any one of *—H, *—F, *—Cl, *—OCF₃, *—CF₃, *—CH₂F, or *—CHF₂, where “*” indicates a point of attachment.

This application claims priority to Korean Patent Application No. 10-2016-0004897 filed on Jan. 14, 2016 and Korean Patent Application No. 10-2016-0044665, filed on Apr. 12, 2016, and all the benefits accruing therefrom under 35 U.S.C. §119, the contents of which in their entirety are herein incorporated by reference.

### BACKGROUND

1. Field

The present invention relates to a liquid crystal composition and a liquid crystal display device including the same.

2. Description of the Related Art

A liquid crystal display device, which is a type of widely used display devices, includes two substrates provided with field generating electrodes, such as a pixel electrode and a common electrode, and a liquid crystal layer disposed between the two substrates.

With the expansion in the field of the liquid crystal display device, it is desirable to improve operating characteristics of the liquid crystal display device, such as response speed, contrast, drive voltage, and the like. In order to improve these characteristics, it is desirable for the liquid crystal compound contained in a liquid crystal composition to have low rotational viscosity, high chemical and physical stability, high liquid crystal phase-isotropic phase transition temperature, low liquid crystal phase lower limit temperature, appropriate elastic modulus, and the like.

### SUMMARY

Aspects of the present invention provide a low-viscosity liquid crystal composition having high reliability and a liquid crystal display device including the same.

According to an exemplary embodiment, a liquid crystal composition is provided. The liquid crystal composition, includes: at least one of compounds represented by Formula 1 below:

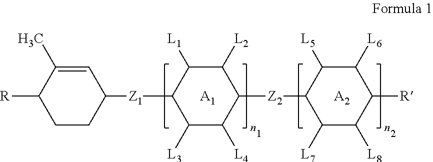

Where in the Formula 1, R—* and R′—* are each independently any one of *—H, *—F, *—Cl, *—I, *—Br, a C₁₋₁₂ alkyl group of C₁₋₁₂, a C₁₋₁₂ alkoxy group, or a cyano group; *—Z₁—* and *—Z₂—* are each independently any one of *—COO—*, *—OCO—*, *—CF₂O—*, *—OCF₂—*, *—CH₂O—*, *—OCH₂—*, *—SCH₂—*, *—CH₂S—*, *—C₂F₄—*, *—CH₂CF₂—*, *—CF₂CH₂—*, *—(CH₂)ₘ—*, *—CH═CH—*, *—CF═CF—*, *—CH═CF—*, *—CF═CH—*, *—C≡C—*, *—CH═CHCH₂O—*, or a single bond;

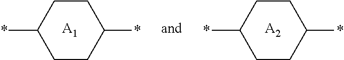

are each independently any one of

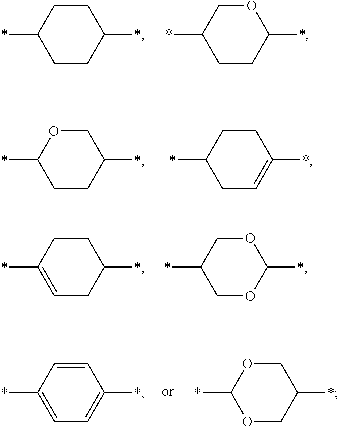

n₁ and n₂ are each independently 0 to 3; m is 2 to 5; and L₁-*, L₂-*, L₃-*, L₄-*, L₅-*, L₆-*, L₇-*, and L₈-* are each independently any one of *—H, *—F, *—Cl, *—OCF₃, *—CF₃, *—CH₂F, or *—CHF₂, where “*” indicates a point of attachment.

In an exemplary embodiment, at least one of the compounds represented by Formula 1 may be any one of compounds represented by Formulae 1-1 to 1-10 below:

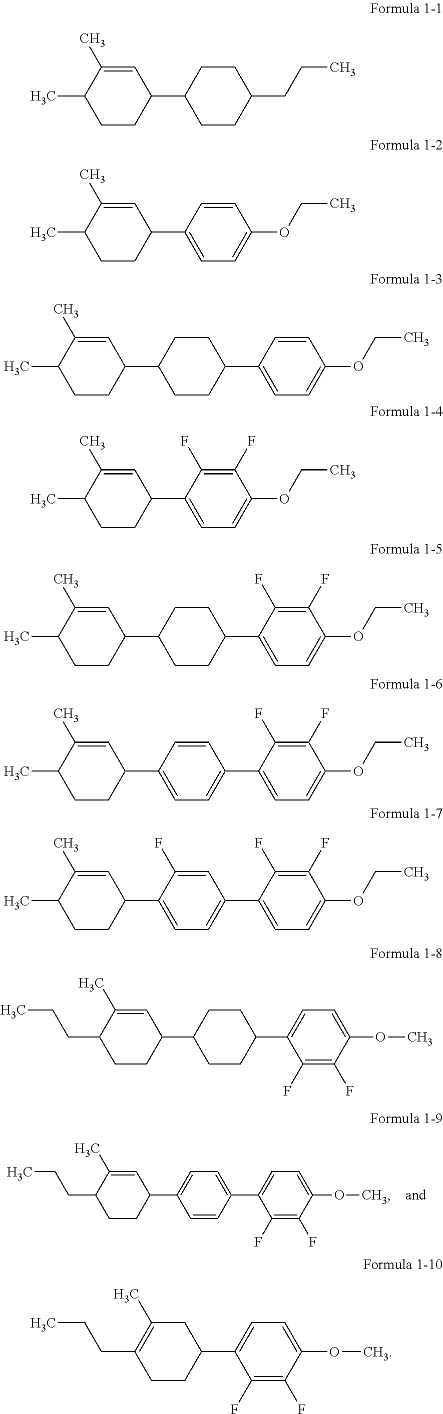

In an exemplary embodiment, the composition may further comprising: at least one of compounds represented by Formulae 2-1 to 2-19 below:

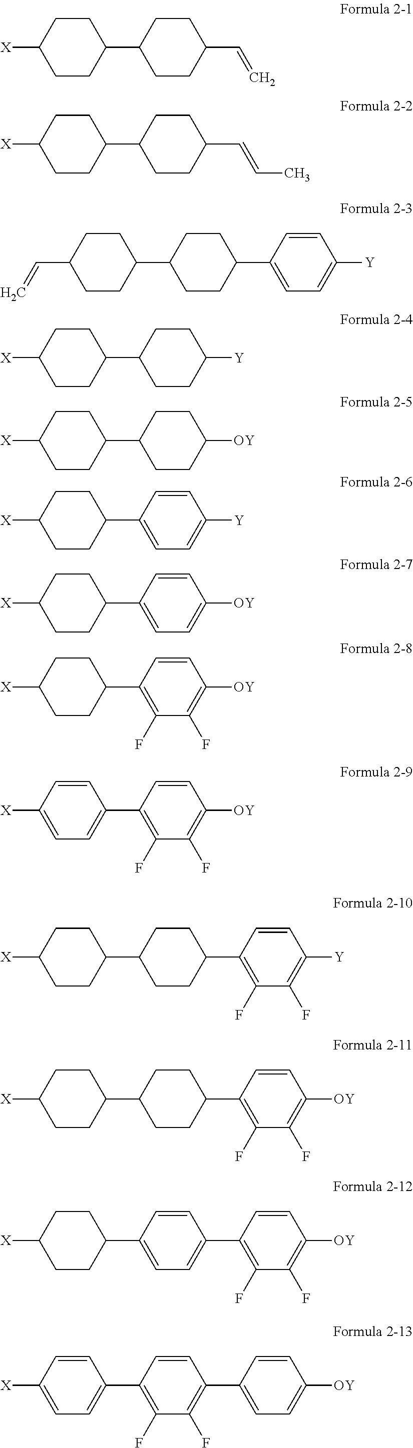

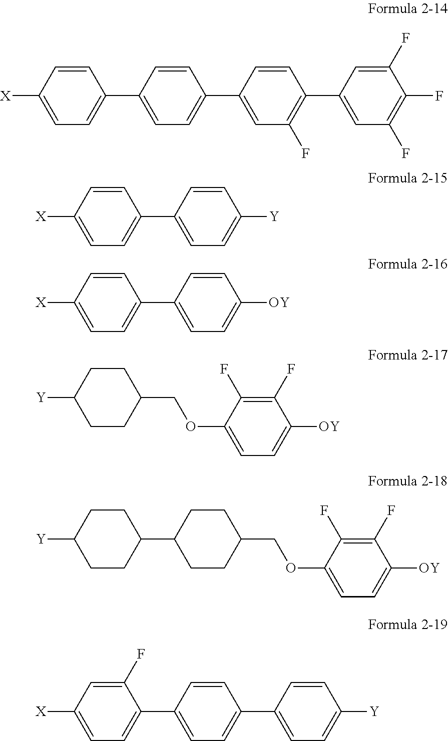

Where in the Formulae 2-1 to 2-19, X—* and Y—* are each independently a C₁₋₅*-alkyl group.

In an exemplary embodiment, the content of a compound represented by Formula P below may be 0 weight percent (wt %).

CH₂═CH—(CH₂)ᵣ-(Cyc)ₛ-(PheF₂)ₜ-R₃₁ Formula P

Where in the Formula P, *-Cyc-* is a 1,4-cyclohexylene group; *-PheF₂-* is a 2,3-fluoro-1,4-phenylene group; r is an integer of 0 to 5; each of s and t is an integer of 0 to 3; and a sum of s and t is an integer of 2 to 4; and *—R₃₁ is a C₁₋₅ alkyl group or a C₁₋₅ alkoxy group.

In an exemplary embodiment, the liquid crystal composition may have a refractive index anisotropy (Δn) of about 0.08 to about 0.12, a dielectric anisotropy (Δ∈) of about −2.8 to about −5.5, and a rotational viscosity (γ1, 20° C.) of about 70 millipascal seconds (mPa·s) to about 140 mPa·s.

According to an exemplary embodiment, a liquid crystal display device is provided. The liquid crystal display device, includes: a display panel including a first base substrate, a switching element disposed on the first base substrate, and a first electrode disposed on the switching element; a counter display panel including a second base substrate and a second electrode disposed on the second base substrate, the counter display panel facing the display panel; and a liquid crystal layer disposed between the display panel and the counter display panel, wherein the liquid crystal layer, wherein the liquid crystal layer includes at least one of compounds represented by Formula 1 and a reactive mesogen including at least one of compounds represented by Formula RM below:

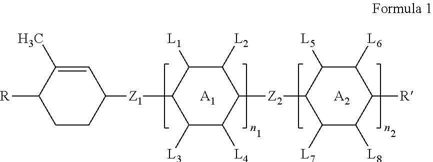

Where in the Formula 1, R—* and R′—* are each independently any one of *—H, *—F, *—Cl, *—I, *—Br, a C₁₋₁₂ alkyl group, a C₁₋₁₂ alkoxy group, or a cyano group; *—Z₁—* and *—Z₂—* are each independently any one of *—COO—*, *—OCO—*, *—CF₂O—*, *—OCF₂—*, *—CH₂O—*, *—OCH₂—*, *—SCH₂—*, *—CH₂S—*, *—C₂F₄—*, *—CH₂CF₂—*, *—CF₂CH₂—*, *—(CH₂)ₘ—*, *—CH═CH—*, *—CF═CF—*, *—CH═CF—*, *—CF═CH—*, *—C≡C—*, *—CH═CHCH₂O—*, or a single bond;

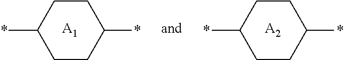

and are each independently any one of

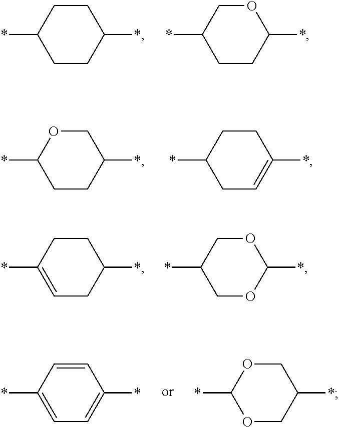

n₁ and n₂ are each independently 0 to 3; m is 2 to 5; and L₁-*, L₂-*, L₃-*, L₄-*, L₅-*, L₆-*, L₇-*, and L₈-* are each independently any one of *—H, *—F, *—Cl, *—OCF₃, *—CF₃, *—CH₂F, or *—CHF₂, where “*” indicates a point of attachment;

P1-SP1-MG-SP2-P2 Formula RM

In the Formula RM, P1-* and P2-* may be each independently any one of

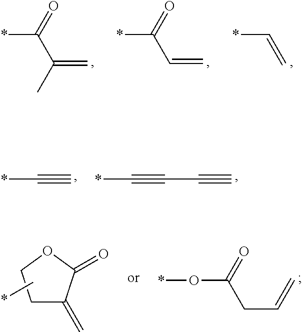

*-SP1-* is *L-Z-L-Arₐ-L-* , where a is 0 to 2, *-SP2-* is *-LAr-L-Z-L_{b}*, where b is 0 to 2; *-L-* is any one of *—(CH₂)_{c}—*, *—O(CH₂)_{c}—*,

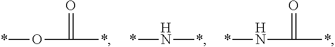

*—CH═CH—*, or *—C≡C—*, *—Z—* is *(CH₂)_{c}—*, and c is 0 to 12; and *—Ar—* is any one of

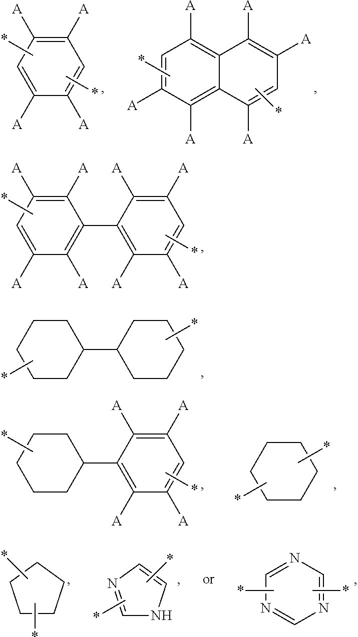

### *-MG-* is

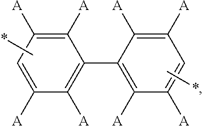

and A-* is any one of H—*, C₁₋₁₀ alkyl, F—*, Br—*, I—*, *—OH, *—NH₂, or *—CN.

In an exemplary embodiment, the liquid crystal display device may further include: a liquid crystal alignment layer disposed between the liquid crystal layer and at least one of the display panel and the counter display panel, and including a polymer of the reactive mesogen.

In an exemplary embodiment, at least one of the compounds represented by Formula 1 may be any one of compounds represented by Formulae 1-1 to 1-10 below:

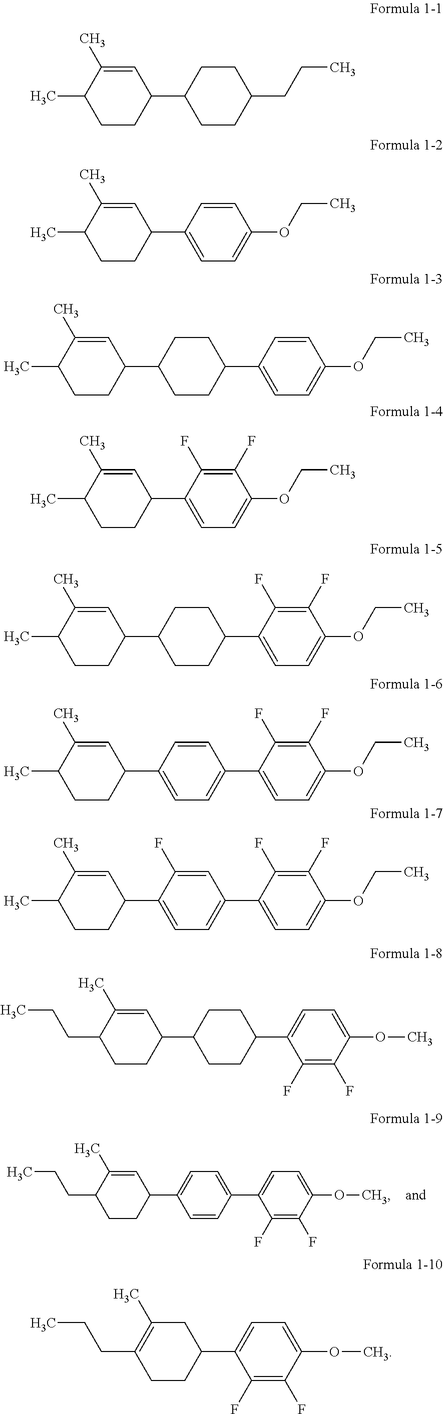

In an exemplary embodiment, the liquid crystal layer may further comprise at least one of compounds represented by Formulae 2-1 to 2-19 below:

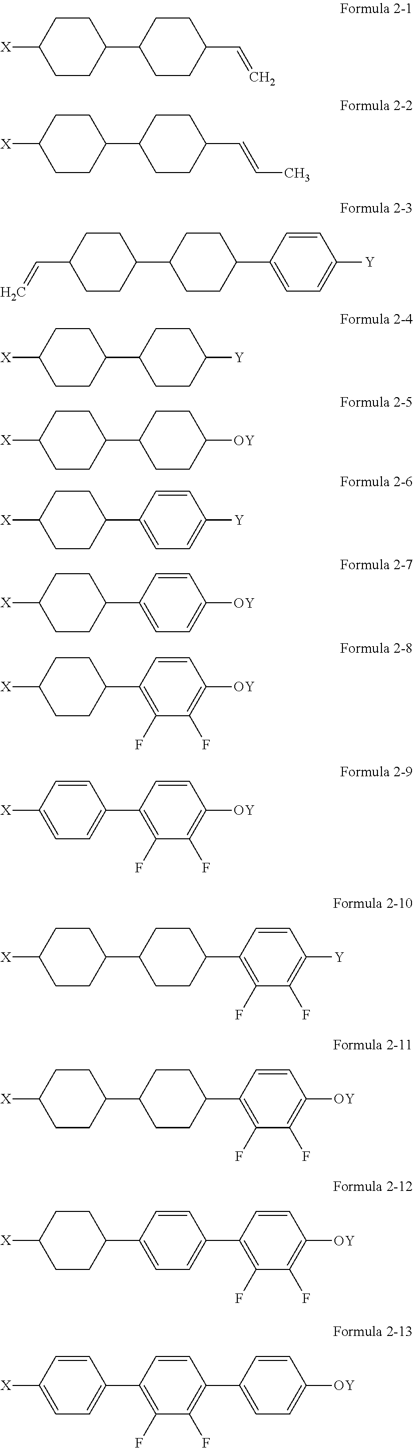

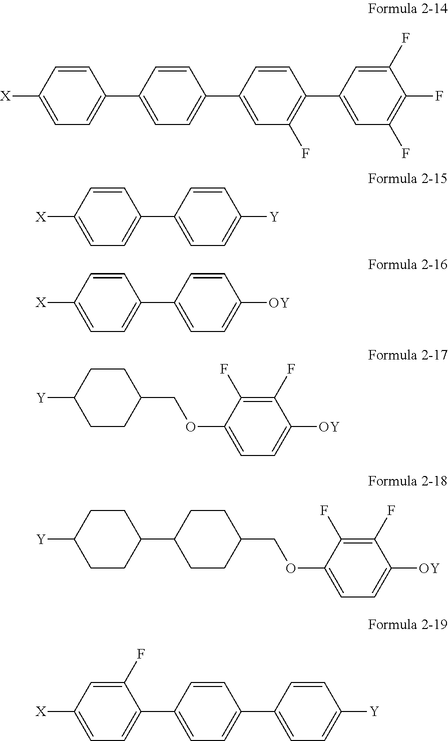

Where in the Formulae 2-1 to 2-19, X—* and Y—* are each independently a C₁₋₅ alkyl group of.

In an exemplary embodiment, the content of a compound represented by Formula P below may be 0 wt %:

CH₂═CH—(CH₂)ᵣ-(Cyc)ₛ-(PheF₂)ₜ-R₃₁ Formula P

In the Formula P, *-Cyc-* is a 1,4-cyclohexylene group; *-PheF₂-* is a 2,3-fluoro-1,4-phenylene group; r is an integer of 0 to 5; each of s and t is an integer of 0 to 3; the sum of s and t is an integer of 2 to 4; and *—R₃₁ is a C₁₋₅ alkyl group or a C₁₋₅ alkoxy group.

In an exemplary embodiment, the liquid crystal display device may further include: a light-blocking spacer disposed between the display panel and the liquid crystal alignment layer, wherein the light-blocking spacer includes a region disposed to overlap the switching element, and wherein a thickness of the liquid crystal layer is determined by a height of the light-blocking layer.

Aspects of the present invention are not restricted to the one set forth herein. The above and other aspects of the present invention will become more apparent to one of ordinary skill in the art to which the present invention pertains by referencing the detailed description of the present invention given below.

### BRIEF DESCRIPTION OF THE DRAWINGS

The above and other aspects and features of the present invention will become more apparent by describing in detail exemplary embodiments thereof with reference to the attached drawings, in which:

FIG. 1 is a schematic exploded perspective view of a first liquid crystal display device;

FIG. 2 is a schematic cross-sectional view of the initial state of the first liquid crystal display device of FIG. 1, when an electric field is not applied;

FIG. 3 is a graph comparing the voltage holding rates (VHR) for the liquid crystal compositions of the Examples (EX1, EX2, and EX3) of Examples with the voltage holding rate of the liquid crystal composition of the Comparative Example (Ref.);

FIG. 4 is a schematic cross-sectional view of the initial state of a second liquid crystal display device, to which an electric field is not applied; and

FIG. 5 is a schematic cross-sectional view of the initial state of a third liquid crystal display device, to which an electric field is not applied.

### DETAILED DESCRIPTION

Features of the invention and methods of accomplishing the same may be understood more readily by reference to the following detailed description of preferred embodiments and the accompanying drawings.

The invention may, however, be embodied in many different forms and should not be construed as being limited to the embodiments set forth herein. Rather, these embodiments are provided so that this disclosure will be thorough and complete and will fully convey the concept of the invention to those skilled in the art, and the invention will only be defined by the appended claims.

In the drawings, the thickness of layers and regions are exaggerated for clarity. It will be understood that when an element or layer is referred to as being “on,” “connected to” or “coupled to” another element or layer, the element or layer can be directly on, connected or coupled to another element or layer or intervening elements or layers. In contrast, when an element is referred to as being “directly on,” “directly connected to” or “directly coupled to” another element or layer, there are no intervening elements or layers present. As used herein, connected may refer to elements being physically, electrically and/or fluidly connected to each other.

Like numbers refer to like elements throughout. As used herein, the term “and/or” includes any and all combinations of one or more of the associated listed items.

It will be understood that, although the terms first, second, third, etc., may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer, or section discussed below could be termed a second element, component, region, layer, or section without departing from the teachings of the invention.

Spatially relative terms, such as “below,” “lower,” “under,” “above,” “upper” and the like, may be used herein for ease of description to describe the relationship of one element or feature to another element(s) or feature(s) as illustrated in the figures. It will be understood that the spatially relative terms are intended to encompass different orientations of the device in use or operation, in addition to the orientation depicted in the figures. For example, if the device in the figures is turned over, elements described as “below” or “beneath” relative to other elements or features would then be oriented “above” relative to the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting. As used herein, the singular forms “a,” “an” and “the” are intended to include the plural forms as well, including “at least one,” unless the context clearly indicates otherwise. It will be further understood that the terms “comprises,” “comprising,” “includes” and/or “including,” when used in this specification, specify the presence of stated features, integers, operations, elements, and/or components, but do not preclude the presence or addition of one or more other features, integers, steps, operations, elements, components, and/or groups thereof. “At least one” is not to be construed as limiting “a” or “an.” “Or” means “and/or.” As used herein, the term “and/or” includes any and all combinations of one or more of the associated listed items.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10%, 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the claims.

In the present specification, the “C_{A-B}” means that the number of carbon atoms (C) is A to B. In the present specification, the symbol “-*” indicates a point of attachment (e.g. a bonding site).

Hereinafter, preferred embodiments will be described in detail with reference to the attached drawings.

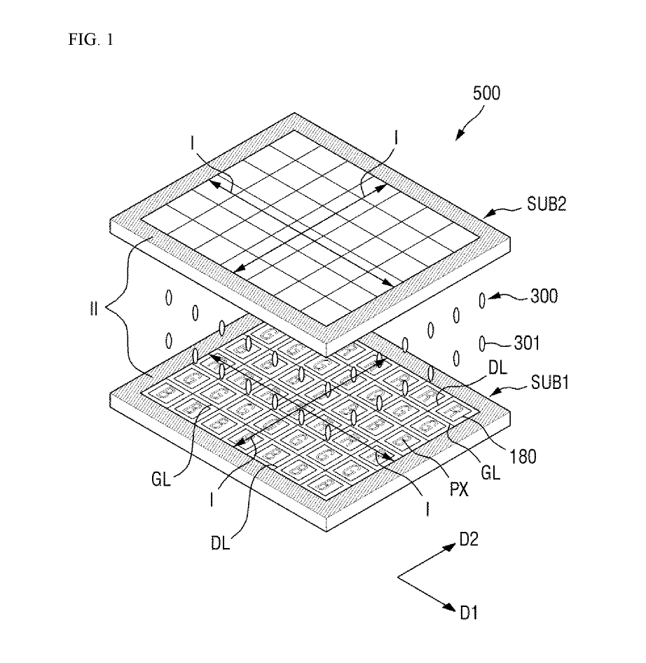

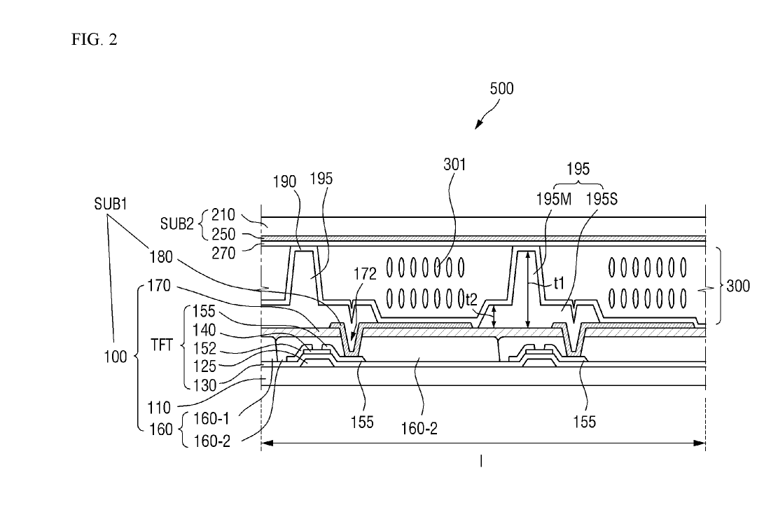

FIG. 1 is a schematic exploded perspective view of a liquid crystal display device 500 including a display panel SUB1 and a counter display panel SUB2, and FIG. 2 is a schematic partial cross-sectional view of the display area I of the liquid crystal display device 500 of FIG. 1.

Referring to FIG. 1, the liquid crystal display device 500 may be configured to include: a display panel SUB1; a counter display panel SUB2 disposed to face the display panel SUB1 and to be spaced apart from the display panel SUB1 while maintaining a predetermined distance; and a liquid crystal layer 300 disposed between the display panel SUB1 and the counter display panel SUB2. The liquid crystal layer 300 may include liquid crystal compound molecules 301, and the liquid crystal compound molecules 301 may have negative dielectric anisotropy.

The liquid crystal display device 500 includes a display area I and a non-display area II. The display area I is an area in which an image is displayed. The non-display area II is a peripheral area surronding the display area I, and is an area in which an image is not displayed.

The display panel SUB1 may include a plurality of gate lines GL extending in a first direction and a plurality of data line DL extending in a second direction perpendicular to the first direction. Although not shown in the drawings, the gate lines GL are not disposed only in the display area I, and may extend to the non-display area II. In this case, the non-display area II may be provided with a gate pad (not shown). That is, in the non-display area II, the display panel SUB1 may include a gate pad (not shown). Further, the data lines DL are not disposed only in the display area I, and may extend to the non-display area II. In this case, the non-display area II may be provided with a data pad (not shown). That is, in the non-display area II, the display panel SUB1 may include a data pad (not shown).

A plurality of pixels PX defined by the gate lines GL the data lines DL may be disposed in the display area I. The plurality of pixels PX may be arranged in the form of a matrix, and a pixel electrode 180 may be disposed for each of the pixels PX. In this case, in the display area I, the display panel SUB1 may include a plurality of pixels PX arranged in the form of a matrix and the plurality of pixel electrodes 180 arranged in the form of a matrix.

In the non-display area II, a drive unit (not shown) for providing a gate drive signal and a data drive signal to each of the pixels PX may be disposed. In this case, in the non-display area II, the display panel SUB1 may include a drive unit (not shown). The drive unit (not shown) may generate a gate drive signal and a data drive signal corresponding to a drive frequency of 120 Hz or more.

The display panel SUB1 may include a switching element array substrate (not shown) and a pixel electrode (not shown), and the counter display panel SUB2 may include a second base substrate (not shown) and common electrode (not shown).

Hereinafter, the display panel SUB1, the counter display panel SUB2, and the liquid crystal layer 300 will be described in more detail with reference to FIGS. 1 and 2.

Referring to FIGS. 1 and 2, the display panel SUB1 may be configured to include a switching element array substrate 100 and a pixel electrode 180. The switching element array substrate 100 may be configured to include a first base substrate 110, a switching element TFT disposed on the first base substrate 110, a color filter layer 160 disposed on the switching element TFT, and an organic film 170 disposed on the color filter layer 160.

The counter display panel SUB2, which is a counter panel of the display panel SUB1, may be configured to include a second base substrate 210 and a common electrode 250.

The liquid crystal display device 500 may further include a light-blocking spacer 195, a first liquid crystal alignment layer 190, and a second liquid crystal alignment layer 270. The light-blocking spacer 195 may be disposed between the pixel electrode 180 and the common electrode 250, and may include an area overlapping the switching element TFT. The light-blocking spacer 195 serves as both a spacer for maintaining the thickness of the liquid crystal layer 300 and a black matrix. The first liquid crystal alignment layer 190 may be disposed on the display panel SUB1, and may include an area disposed between the pixel electrode 180 and the liquid crystal layer 300. The second liquid crystal alignment layer 270 may include an area disposed between the common electrode 250 and the liquid crystal layer 300. Further, the first liquid crystal alignment layer 190 may include an area disposed between the light-blocking spacer 195 and the second liquid crystal alignment layer 270, and the second liquid crystal alignment layer 270 may include an area disposed between the first liquid crystal alignment layer 190 and the common electrode 250.

The liquid crystal display device 500 may be a polymer stabilized-vertical alignment mode (PS-VA mode) type. The PS-VA mode is a technology for stabilizing the pretilt alignment of liquid crystal compound molecules 301 through a polymer network composed of polymers of reactive mesogens. The PS-VA mode may be manufactured by a first method in which the liquid crystal layer 300 is formed using a liquid crystal composition containing the reactive mesogens, and then a polymer network composed of polymers of the reactive mesogens is formed through an ultraviolet exposure process, or a second method in which a liquid crystal aligning agent containing the reactive mesogens is applied onto at least one electric field generating electrode of the pixel electrode 180 and the common electrode 250 to form a film, the reactive mesogens are eluted into the liquid crystal layer 300, and then a polymer network composed of polymers of the reactive mesogens is formed through an ultraviolet exposure process.

The reactive mesogen is a compound having a mesogenic structure for expressing liquid crystallinity and a polymerizable end group for polymerization. For example, the reactive mesogen may be represented by Formula RM below.

P1-SP1-MG-SP2-P2 Formula RM

In the Formula RM, P1-* and P2-* may be each independently any one of

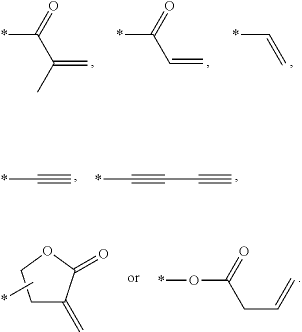

In the Formula RM, *-SP1-* may be *L-Z-L-ArₐL-*, a may be 0 to 2, *-SP2-* may be *-LAr-L-Z-L_{b}-*, and b may be 0 to 2.

In the Formula RM, *-L-* may be any one of *—(CH₂)_{c}—*, *—O(CH₂)_{c}—*,

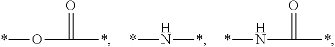

*—CH═CH—*, or *—C≡C—*, *—Z—* may be *—(CH₂)_{c}—*, and c may be 0 to 12.

In the Formula RM, *—Ar—* may be any one of

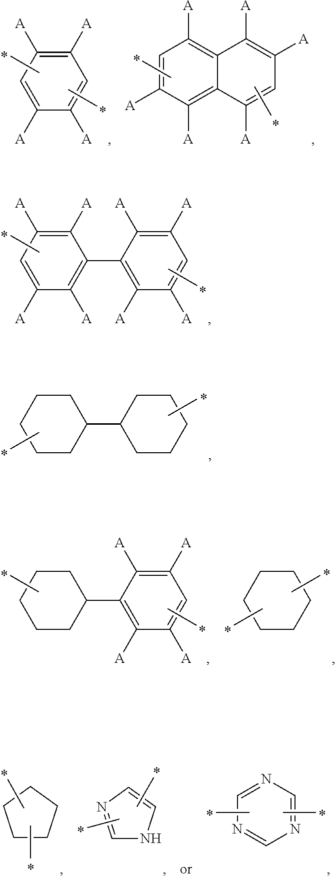

### *-MG-* may be

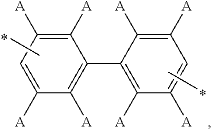

and A-* may be any one of H—*, C₁₋₁₀ alkyl, F—*, Br—*, I—*, *—OH, *—NH₂, or *—CN.

The reactive mesogen, for example, may be at least one of compounds represented by Formula RM1 below and at least one of compounds represented by Formula RM2 below.

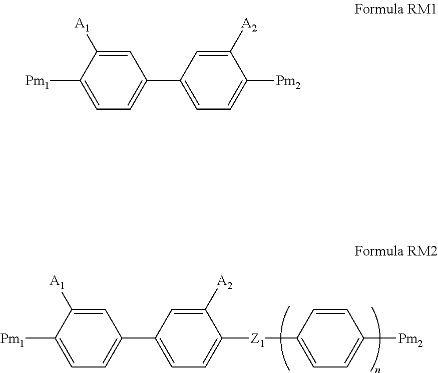

In the Formulae RM1 and RM2, Pm₁-* and Pm₂-* may be each independently any one of

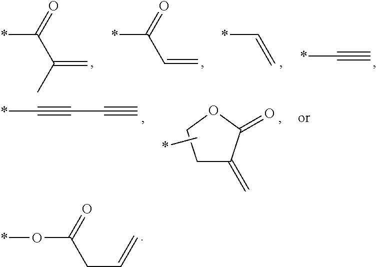

Pm₁-* and Pm₂-* may be the same as or different from each other, and each of A₁-* and A₂-* may be any one of *—H, *—F, *—Br, *—I, *—OH, *—NH₂, or *—CN.

Meanwhile, in the Formula RM2, *—Z₁—* may be any one of *—(CH₂)_{d}—* or *—O(CH₂)_{d}—*, n may be 1 to 2, and d may be 1 to 10.

Since the compound represented by the Formula RM1 has relatively poor thermal stability compared to the compound represented by the Formula RM2, the compound represented by the Formula RM1 is easily deteriorated in the high-temperature heat treatment process for forming the first and second liquid crystal alignment layers 190 and 270. Therefore, it is desirable that the compound represented by the Formula RM1 is added to the liquid crystal composition during the process of manufacturing the liquid crystal display device 500, and the compound represented by the Formula RM2 is added to the liquid crystal aligning agent during the process of manufacturing the liquid crystal display device 500.

Meanwhile, both the first method and the second method require an ultraviolet light exposure process for polymerizing the reactive mesogens. Therefore, it is preferred that the liquid crystal layer 300 is composed of liquid crystal compound molecules 301 having excellent photostability.

The compound represented by Formula P below is a low-viscosity liquid crystal compound generally used to improve the high-speed response characteristics of the liquid crystal display device 500, because this compound has excellent rotational viscosity, elastic modulus and phase transition temperature (Tni) compared to liquid crystal compounds each having a terminal alkyl group at the end, for example, liquid crystal compounds having a structure in which a terminal alkyl group is present instead of CH₂═CH— in Formula P below. However, this compound represented by Formula is disadvantageous in that photostability is very poor due to the presence of the terminal double bond.

CH₂═CH—(CH₂)ᵣ-(Cyc)ₛ-(PheF₂)ₜ-R₃₁ Formula P

In the Formula P, *-Cyc-* may be a 1,4-cyclohexylene group, and *-PheF₂-* may be a 2,3-fluoro-1,4-phenylene group. In the Formula P, r may be an integer of 0 to 5, each of s and t may be an integer of 0 to 3 and the sum of s and t may be an integer of 2 to 4, and *—R₃₁ may be a C₁₋₅ alkyl group or a C₁₋₅ alkoxy group.

For example, the compound represented by Formula P below may be any one of compound represented by Formulae P-1 to P-4.

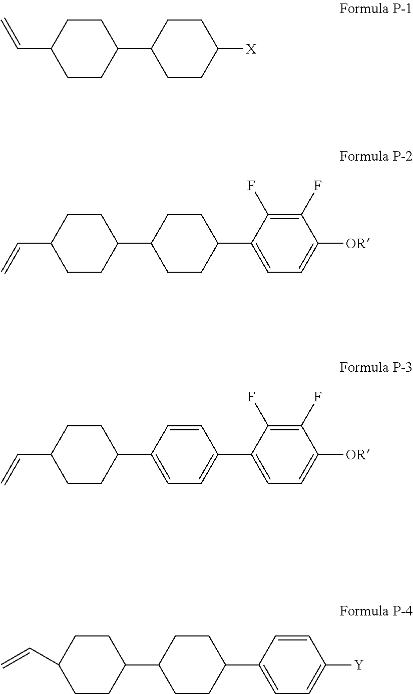

In the Formulae P-1 to P-4, X—*, Y—*, and R′—* are each independently a C₁₋₅ alkyl group.

As described above, the counter display panel SUB2 is a counter panel of the display panel SUB1, and includes the second base substrate 210 and the common electrode 250. In this case, the common electrode 250 may be directly disposed on the second base substrate 210. In this case, the liquid crystal display device 500 may be configured such that the display panel SUB1 includes the color filter layer 160 and the light-blocking spacer 195, and the counter display panel SUB2 does not include a color filter and a black matrix. In this case, at the time of the ultraviolet exposure process, the exposure amount of ultraviolet light incident on the liquid crystal layer 300 increases, compared to the case where the counter display panel SUB2 is designed to have a structure including a color filter and a black matrix.

Therefore, when the liquid crystal layer 300 contains the compound represented by the Formula P, the compound represented by the Formula P is easily deteriorated, and thus the voltage holding rate (VHR) of the liquid crystal display device 500 may be lowered. For this reason, it is desirable that the liquid crystal layer 300 does not contain the compound represented by the Formula P.

Meanwhile, the light-blocking spacer 195 may include a photosensitizing agent, a black pigment, a binder, a multifunctional monomer, a photoinitiator, and a solvent. The black pigment may be a mixture of a blue pigment, a red pigment, and a yellow pigment, or may be a lactam-based black pigment. The blue pigment may be a compound represented by Formula B below, the red pigment may be a compound represented by Formula R below, and the yellow pigment may be a compound represented by Formula Y below. The lactam-based black pigment may be a compound represented by Formula L below.

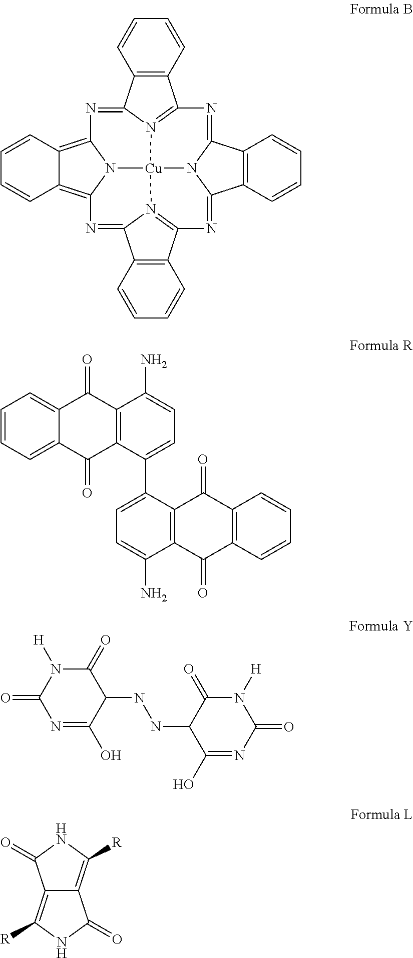

In the Formula L, R—* may be a C₁₋₁₀ hydrocarbon group or a derivative of a C₁₋₁₀ hydrocarbon group.

The binder may be any one of compounds represented by Formulae BD1 and BD2.

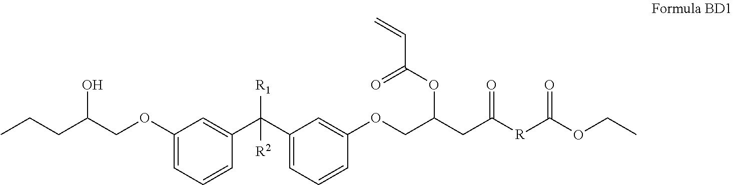

In the Formula BD1, R₁—* and R₂—* may be each independently a C₁₋₁₀ hydrocarbon group or a derivative of a C₁₋₁₀ hydrocarbon group, and *—R—* may be a C₁₋₁₀ alkylene.

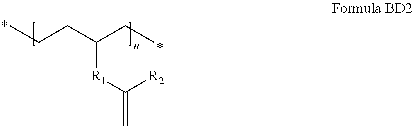

In the Formula BD2, *—R₁—* may be a C₁₋₁₀ alkylene group, R₂—* may be a C₁₋₁₀ hydrocarbon group or a derivative of a C₁₋₁₀ hydrocarbon group, and n is an integer of 1 to 100.

For example, the light-blocking spacer 195 may be formed by applying the photosensitizing agent onto the display panel SUB1 or the counter display panel SUB2 and exposing and developing the applied photosensitizing agent. At this time, unreacted components may be introduced into the first and second liquid crystal alignment layers 190 and 270. The unreacted components may be residues of at least one of the black pigment and the binder.

Since the unreacted components are not completely removed, they may damage the liquid crystal compound molecules 301 at the time of the ultraviolet light exposure. In particular, the compound represented by the Formula P is vulnerable to the unreacted components.

For this reason, it is desirable that the liquid crystal layer 300 does not contain the compound represented by the Formula P.

The liquid crystal layer 300 includes a liquid crystal composition containing at least one of compounds represented by Formula 1 below instead of the compound represented by the Formula P.

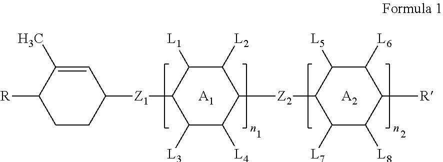

In the Formula 1, R—* and R′—* are each independently any one of *—H, *—F, *—Cl, *—I, *—Br, a C₁₋₁₂ alkyl group, a C₁₋₁₂ alkoxy group, or a cyano group. In the Formula 1, *—Z₁—* and *—Z₂-* are each independently any one of *—COO—*, *—OCO—*, *—CF₂O—*, *—OCF₂—*, *—CH₂O—*, *—OCH₂—*, *—SCH₂—*, *—CH₂S—*, *—C₂F₄—*, *—CH₂CF₂—*, *—CF₂CH₂—*, *—(CH₂)ₘ—*, *—CH═CH—*, *—CF═CF—*, *—CH═CF—*, *—CF═CH—*, *—C≡C—*, *—CH═CHCH₂O—*, or a single bond.

In the Formula 1,

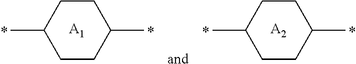

and are each independently any one of

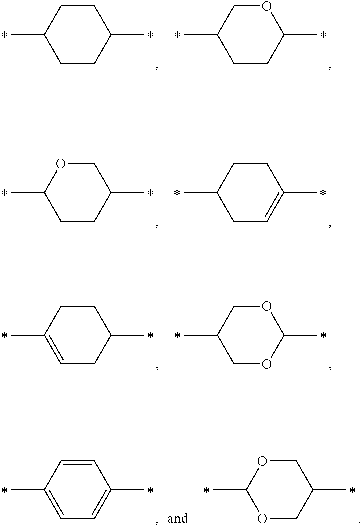

In the Formula 1, n₁ and n₂ are each independently 0 to 3, m is 2 to 5, and L₁-*, L₂-*, L₃-*, L₄-*, L₅-*, L₆-*, L₇-*, and L₈-* are each independently any one of *—H, *—F, *—Cl, *—OCF₃, *—CF₃, *—CH₂F, or *—CHF₂.

The compound represented by the Formula 1 above has an advantage of relatively excellent photostability as compared to the compound represented by the Formula P above, because the end groups R—* and R′—* do not include double bonds and the core structure includes 1,3,6-trimethylcyclohexene. Therefore, the compound represented by the Formula 1 above may improve the voltage holding rate of the liquid crystal display device 500. Further, the compound represented by the Formula 1 above may also improve the high-speed response characteristics of the liquid crystal display device 500 because the compound has low-viscosity characteristics.

The compound represented by the Formula 1, for example, may be any one of compounds represented by Formulae 1-1 to 1-10.

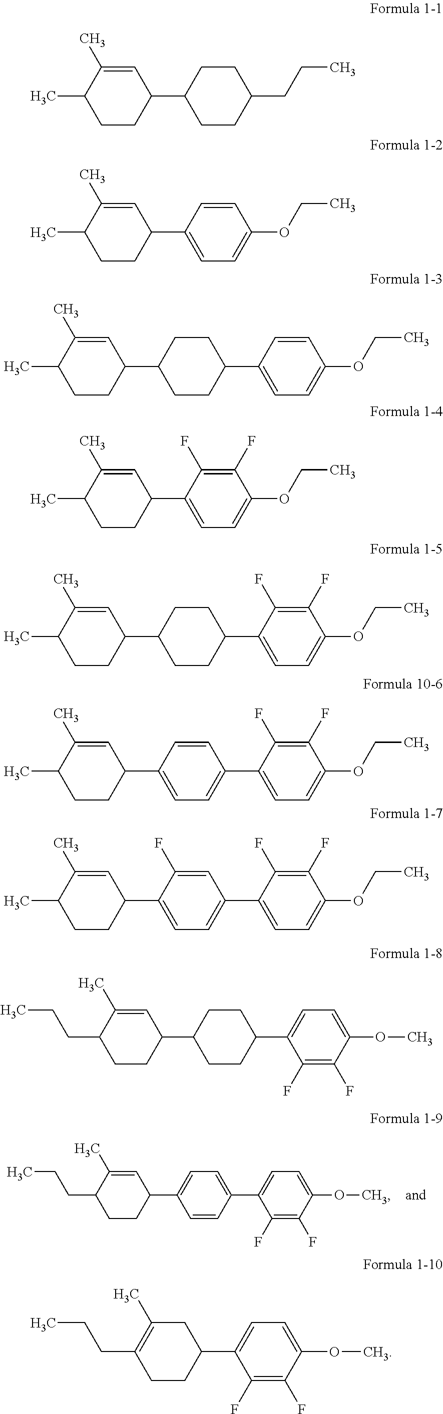

The simulation results of the phase transition temperature (Tni), refractive index anisotropy (Δn), dielectric anisotropy (Δ∈), and rotational viscosity (γ1) of the compounds 136T1, 136T2, 136T3, 136T4, 136T5, 136T6, and 136T7 represented by the Formulae 1-1 to 1-7, respectively, are summarized in Table 1 below.

TABLE 1

| Liquid | | | | | |
|---|---|---|---|---|---|
| crystal | | Tni | Δε | Δn | γ1 |
| compound | Structural Forlula | (° C.) | (ε \|\| − ε⊥) | (ne − no) | (mPa · s) |
| 136T1 | 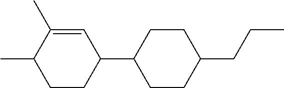 [SMILES: CCCC1CCC(C2C=C(C)C(C)CC2)CC1] | 72.4 | −0.3988 | 0.0782 | 48 |
| 136T2 | 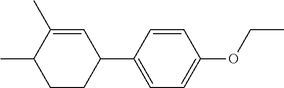 [SMILES: CCOc1ccc(C2C=C(C)C(C)CC2)cc1] | 70.9 | −0.4682 | 0.1366 | 42 |
| 136T3 | 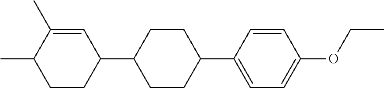 [SMILES: CCOc1ccc(C2CCC(C3C=C(C)C(C)CC3)CC2)cc1] | 98.2 | −2.7288 | 0.1205 | 230 |
| 136T4 | 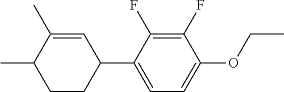 [SMILES: CCOc1ccc(C2C=C(C)C(C)CC2)c(F)c1F] | 75.8 | −4.0167 | 0.1331 | 78 |
| 136T5 | 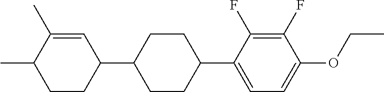 [SMILES: CCOc1ccc(C2CCC(C3C=C(C)C(C)CC3)CC2)c(F)c1F] | 104.4 | −3.801 | 0.115 | 397 |
| 136T6 | 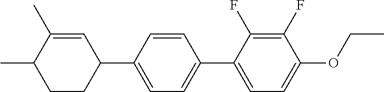 [SMILES: CCOc1ccc(-c2ccc(C3C=C(C)C(C)CC3)cc2)c(F)c1F] | 111.8 | −3.806 | 0.208 | 410 |
| 136T7 | 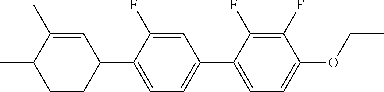 [SMILES: CCOc1ccc(-c2ccc(C3C=C(C)C(C)CC3)c(F)c2)c(F)c1F] | 111.8 | −5.227 | 0.171 | 466 |

The results of measurement of the phase transition temperature (Tni), refractive index anisotropy (Δn), dielectric anisotropy (Δ∈), elastic modulus (K33), and rotational viscosity (γ1) of the liquid crystal compositions of Comparative Examples not containing the compounds 136T1, 136T2, 136T3, 136T4, 136T5, 136T6, and 136T7 represented by the Formulae 1-1 to 1-7, respectively, are summarized in Table 2 below.

The results of measurement of the phase transition temperature (Tni), refractive index anisotropy (Δn), dielectric anisotropy (Δ∈), elastic modulus (K33) and rotational viscosity (γ1) of the liquid crystal compositions of Examples containing the compounds 136T1, 136T2, 136T3, 136T4, 136T6, and 137T7 represented by the Formulae 1-1, 1-2, 1-3, 1-4, 1-6, and 1-7, respectively, are summarized in Table 3 below.

The results of measurement of the phase transition temperature (Tni), refractive index anisotropy (Δn), dielectric anisotropy (Δ∈), elastic modulus (K33) and rotational viscosity (γ1) of the liquid crystal compositions of Examples containing the compounds 136T2, 136T4, 136T5, and 137T7 represented by the Formulae 1-2, 1-4, 1-5, and 1-7, respectively, are summarized in Table 4 below.

The results of measurement of the phase transition temperature (Tni), refractive index anisotropy (Δn), dielectric anisotropy (Δ∈), elastic modulus (K33) and rotational viscosity (γ1) of the liquid crystal compositions of Examples containing the compounds 136T1, 136T2, 136T4, 136T5, 136T6, and 137T7 represented by the Formulae 1-1, 1-2, 1-4, 1-5, 1-6, and 1-7, respectively, are summarized in Table 5 below.

In Tables 2 to 5, the structures of the liquid crystal compounds used in each of the Example and Comparative Example liquid crystal compositions are provided using abbreviations for groups in the structure. The amount of liquid crystal compound (wt %) in the liquid crystal composition is also provided. In Tables 2 to 5,

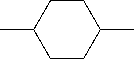

is named as “C”,

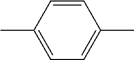

is named as “P”,

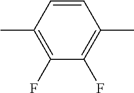

is named as “A”,

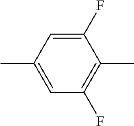

is named as “K”,

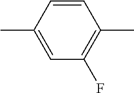

is named as “L”,

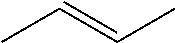

is named as “V”, and

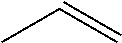

is named as “V1”.

For example,

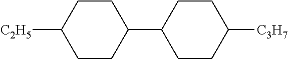

may be named as “2CC3”,

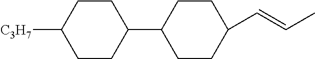

may be names as “3CCV”,

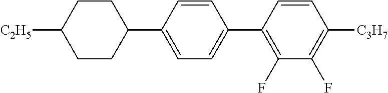

may be names as “2CPA3”,

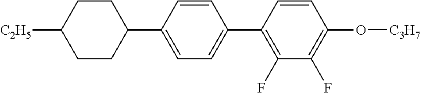

may be named as “2CPAO3”, and

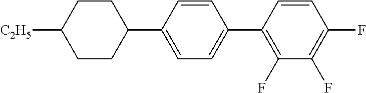 [SMILES: CCC1CCC(c2ccc(-c3ccc(F)c(F)c3F)cc2)CC1]

may be named as “2CPAF”.

TABLE 2

| Comparative | Liquid crystal | | |
|---|---|---|---|
| Example | compound | Content (wt %) | Physical properties |
| 1 | 3CCV | 28.5 | Tni (° C.): 75 |
| 2 | 3CCV1 | 10 | Δn (ne − no): 0.108 |
| 3 | 3CAO4 | 15 | Δε (ε// − ε⊥): −3.0 |
| 4 | 5CAO2 | 6.5 | K33: 15.7 |
| 5 | 3CCAO2 | 11 | γ1 (mPa · s): 97 |
| 6 | 2CPAO2 | 5.5 | |
| 7 | 3CPAO2 | 10.5 | |
| 8 | 2PAP3 | 12.5 | |
| 9 | 3PPLKF | 0.5 | |

TABLE 3

| | Liquid crystal | | |
|---|---|---|---|
| Example | compound | Content (wt %) | Physical properties |
| 1 | 3CCV | 15 | Tni (° C.): 78 |
| 2 | 2CC3 | 5 | Δn (ne − no): 0.108 |
| 3 | 136T1 | 6 | Δε (ε// − ε⊥): −3.0 |
| 4 | 136T2 | 15 | K33: 15.5 |
| 5 | 136T3 | 3 | γ1 (mPa · s): 78 |
| 6 | 136T4 | 3 | |
| 7 | 3CAO2 | 10 | |
| 8 | 3CAO4 | 5 | |
| 9 | 3PAO2 | 16 | |
| 10 | 3CCAO2 | 4 | |
| 11 | 3CCAO3 | 5 | |
| 12 | 3CPAO2 | 4 | |
| 13 | 136T6 | 3 | |
| 14 | 136T7 | 6 | |

Referring to Tables 2 and 3 above, each of the liquid crystal compositions of the Examples does not include 3CCV1 having a double bond at the end thereof or 2PAP3 and 3PPLKF having a terphenyl group at the end thereof. From the results, the liquid crystal compositions of the Examples may ensure high-reliability, low-viscosity characteristics as compared to the liquid crystal compositions of the Comparative Examples.

TABLE 4

| | Liquid crystal | | |
|---|---|---|---|
| Example | compound | Content (wt %) | Physical properties |
| 1 | 3CCV | 15 | Tni (° C.): 74.5 |
| 2 | 3CC4 | 5 | Δn (ne − no): 0.108 |
| 3 | 136T2 | 8 | Δε (ε// − ε⊥): −3.8 |
| 4 | 136T4 | 3 | K33: 15.5 |
| 5 | 3CAO2 | 15 | γ1 (mPa · s): 90 |
| 6 | 3CAO4 | 9 | |
| 7 | 3PAO2 | 17 | |
| 8 | 3CCAO3 | 7 | |
| 9 | 2CPAO2 | 10 | |
| 10 | 2PAP3 | 3 | |
| 11 | 136T5 | 2 | |
| 12 | 136T7 | 6 | |

Referring to Tables 2 and 4 above, the liquid crystal compositions of the Examples were able to ensure high dielectric characteristics as compared to the liquid crystal compositions of the Comparative Examples, because they include a polar liquid crystal compound including 1,3,6-trimethylcyclohexene.

TABLE 5

| | Liquid crystal | | |
|---|---|---|---|
| Example | compound | Content (wt %) | Physical properties |
| 1 | 3CCV | 15 | Tni (° C.): 88.0 |
| 2 | 3CC4 | 9 | Δn (ne − no): 0.108 |
| 3 | 136T1 | 8 | Δε (ε// − ε⊥): −3.3 |
| 4 | 136T2 | 8 | K33: 16.0 |
| 5 | 136T4 | 5 | γ1 (mPa · s): 95 |
| 6 | 3CAO2 | 10 | |
| 7 | 3CAO4 | 5 | |
| 8 | 3PAO2 | 10 | |
| 9 | 3CCAO3 | 5 | |
| 10 | 2CPAO2 | 5.5 | |
| 11 | 3CPAO2 | 10.5 | |
| 12 | 136T5 | 3 | |
| 13 | 136T6 | 3 | |
| 14 | 136T7 | 3 | |

Referring to Tables 2 and 5 above, the liquid crystal compositions of Examples were able to ensure high phase transition temperature and high dielectric characteristics as compared to the liquid crystal compositions of the Comparative Examples, because they use a polar liquid crystal compound including 1,3,6-trimethylcyclohexene.

Meanwhile, the simulation results of the phase transition temperature (Tni), refractive index anisotropy (Δn), dielectric anisotropy (Δ∈), and rotational viscosity (γ1) of the comparative compounds Ref.1, Ref.2, Ref.3, 124T1, 124T2, and 124T3, and the compounds 136T1, 136T2, and 136T3 represented by the Formulae 1-8 to 1-10, respectively, are summarized in Table 6 below.

TABLE 6

| Liquid | | | | | |
|---|---|---|---|---|---|
| crystal | | Tni | Δε | Δn | γ1 |
| compound | Structural Forlula | (° C.) | (ε // − ε⊥) | (ne − no) | (mPa · s) |
| Ref. 1 | 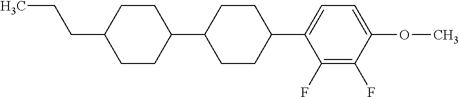 [SMILES: CCCC1CCC(C2CCC(c3ccc(OC)c(F)c3F)CC2)CC1] | 82.2 | −5.4 | 0.087 | 188 |
| 124T1 | 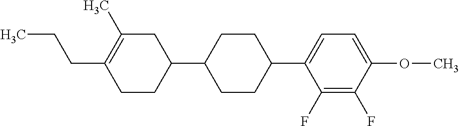 [SMILES: CCCC1=C(C)CC(C2CCC(c3ccc(OC)c(F)c3F)CC2)CC1] | 99.8 | −5.33 | 0.123 | 228 |
| 136T8 | 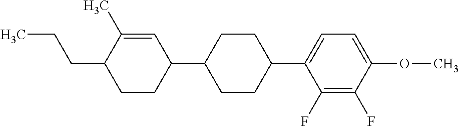 [SMILES: CCCC1CCC(C2CCC(c3ccc(OC)c(F)c3F)CC2)C=C1C] | 97.0 | −5.36 | 0.113 | 193 |
| Ref. 2 | 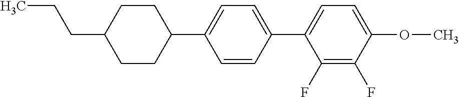 [SMILES: CCCC1CCC(c2ccc(-c3ccc(OC)c(F)c3F)cc2)CC1] | 78.6 | −5.35 | 0.152 | 248 |
| 124T2 | 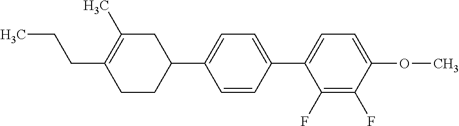 [SMILES: CCCC1=C(C)CC(c2ccc(-c3ccc(OC)c(F)c3F)cc2)CC1] | 94.2 | −5.18 | 0.195 | 310 |
| 136T9 | 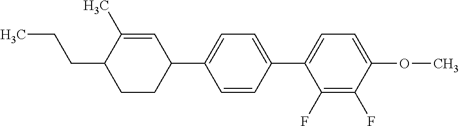 [SMILES: CCCC1CCC(c2ccc(-c3ccc(OC)c(F)c3F)cc2)C=C1C] | 91.5 | −5.22 | 0.182 | 273 |
| Ref. 3 | 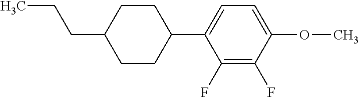 [SMILES: CCCC1CCC(c2ccc(OC)c(F)c2F)CC1] | 64.2 | −5.01 | 0.100 | 46 |
| 124T3 | 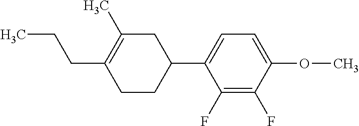 [SMILES: CCCC1=C(C)CC(c2ccc(OC)c(F)c2F)CC1] | 70.5 | −4.89 | 0.149 | 43 |
| 136T10 | 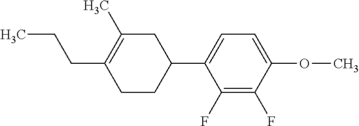 [SMILES: CCCC1=C(C)CC(c2ccc(OC)c(F)c2F)CC1] | 68.6 | −4.89 | 0.135 | 40 |

Referring to Table 6 above, the phase transition temperature and refractive index anisotropy of the compounds (136T8, 136T9, and 136T10) represented by the Formula 1 were able to be improved as compared to those of the comparative compounds (Ref. 1, Ref 2, and Ref.3), and the rotational viscosity thereof could be improved compared to that of the comparative compounds (124T1, 124T2, and 124T3).

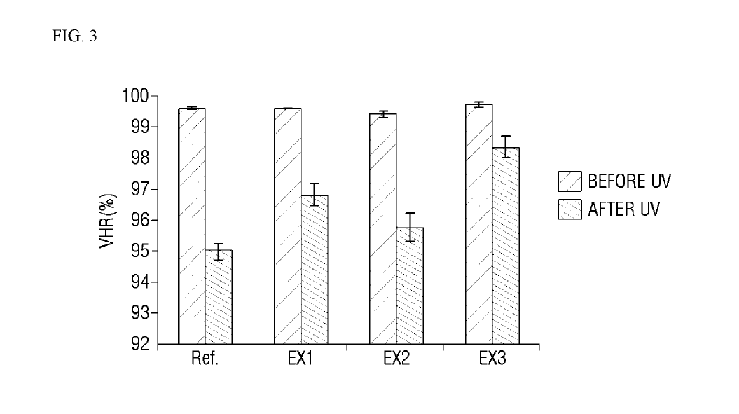

Meanwhile, FIG. 3 shows a graph comparing the measurement results of the voltage holding rates of the liquid crystal compositions EX1, EX2, and EX3 (Tables 3 to 5) with the measurement result of the voltage holding rate of the liquid crystal composition (Ref) of Table 2. The measurement results of the voltage holding rates of FIG. 3 are summarized in Table 7 below. The voltage holding rates thereof were measured under the following conditions.
Frame frequency: 60 hertz (Hz) (16.64 milliseconds (ms))
Pulse width: 64 microseconds (μs)
Data voltage: 1 volt (V)
Cell gap: 3.0 micrometers (μm)
Exposure condition: 6 joules (J), 70 seconds

TABLE 7

| | | Voltage holding rate | | | | | | |
|---|---|---|---|---|---|---|---|---|
| | | (%) | | Average value | | | Standard deviation | |
| | | Before | After | Before | After | Preservation | Before | After |
| | Sample | exposure | exposure | exposure | exposure | rate (%) | exposure | exposure |
| Ref. | 1 | 99.62 | 94.84 | 99.61 | 94.98 | 95.35 | 0.040 | 0.248 |
| | 2 | 99.57 | 95.27 | | | | | |
| | 3 | 99.65 | 94.84 | | | | | |
| EX1 | 1 | 99.59 | 97.20 | 99.61 | 96.82 | 97.20 | 0.026 | 0.337 |
| | 2 | 99.64 | 96.72 | | | | | |
| | 3 | 99.60 | 96.55 | | | | | |
| EX2 | 1 | 99.45 | 95.30 | 99.42 | 95.77 | 96.33 | 0.104 | 0.456 |
| | 2 | 99.50 | 96.21 | | | | | |
| | 3 | 99.30 | 95.80 | | | | | |
| EX3 | 1 | 99.81 | 98.67 | 99.72 | 98.36 | 98.63 | 0.085 | 0.350 |
| | 2 | 99.64 | 98.43 | | | | | |
| | 3 | 99.72 | 97.98 | | | | | |

Referring to FIG. 3 and Table 7, it can be ascertained that the liquid crystal compositions (EX1, EX2, and EX3) of the Examples have high voltage holding rates even after exposure, compared to the liquid crystal composition (Ref) of the Comparative Example.

Referring to FIGS. 1 and 2 again, the first base substrate 110 is a base substrate of the switching element array substrate 100, and may be made of a transparent insulating material, such as glass or transparent plastic.

The switching element TFT may be a thin film transistor, and the thin film transistor may be configured to include a gate electrode 125, a gate insulating film 130, a semiconductor layer 140, a source electrode 152, and a drain electrode 155. The gate electrode 125, which is a control terminal of the thin film transistor, may be disposed on the first base substrate 110, and may be made of a conductive material. The gate electrode 125 may be branched from the gate line GL. The gate insulating film 130 may be disposed between the gate electrode 125 and the semiconductor layer 140 to insulate them, and may be formed to extend from the display area I to the non-display area II. The semiconductor layer 140, which is a channel layer of the thin film transistor, may be disposed on the gate insulating film 130. The source electrode 152 and the drain electrode 155 may be disposed on the semiconductor layer 140 to be spaced from each other, and may be made of a conductive material. The source electrode 152 is an input terminal of the thin film transistor, and the drain electrode 155 is an output terminal of the thin film transistor. The source electrode 152 and the drain electrode 155 may be branched from the data line DL. Ohmic contact layers (not shown) may be respectively formed between the source electrode 152 and the semiconductor layer 140 and between the drain electrode 155 and the semiconductor layer 140.

The gate line GL may be disposed between the first base substrate 110 and the pixel electrode 180, and the data line DL may be disposed between the gate line GL and the pixel electrode 180.

The color filter layer 160 may be formed on the source electrode 152 and the drain electrode 155. The color filter layer 160 may be disposed on the switching element TFT, and, specifically, may be disposed between the switching element TFT and the pixel electrode 180. The color filter layer 160 may be formed in a region corresponding to each pixel PX in the display area I, and includes a first color filter 160-1 and a second color filter 160-2. For example, the first color filter 160-1 and the second color filter 160-2 may be color filters realizing different colors from each other. Each of the first color filter 160-1 and the second color filter 160-2 may be one of a red color filter (R), a green color filter (G), and a blue color filter (B). The first color filter 160-1 and the second color filter 160-2 may be arranged alternately.

The organic film 170 made of an organic material may be formed on the color filter layer 160. The organic film 170 may extend to the non-display area II.

On the organic film 170, the pixel electrode 180 made of a conductive material may be formed for each pixel PX. The pixel electrode 180 may be electrically connected with the drain electrode 155 through a contact hole 172 defined in the organic film 170 and the color filter layer 160. The switching element TFT is electrically connected to the gate line GL and the pixel electrode 180. The pixel electrode 180 may be made of indium tin oxide, indium zinc oxide, indium oxide, zinc oxide, tin oxide, gallium oxide, titanium oxide, aluminum, silver, platinum, chromium, molybdenum, tantalum, niobium, zinc, magnesium, an alloy thereof, or a laminate thereof. The pixel electrode 180 is disposed between the color filter layer 160 and the liquid crystal layer 300.

The pixel electrode 180 forms an electric field together with the common electrode 250 to control the alignment direction of liquid crystal molecules in the liquid crystal layer 300 disposed therebetween. The pixel electrode 180 may be a pattern electrode having at least one of a protrusion pattern and a slit pattern, or may be a patternless electrode.

The light-blocking spacer 195 may be disposed on the display panel SUB1. Specifically, the light-blocking spacer 195 may include an area disposed between the pixel electrode 180 and the common electrode 250, and an area disposed to overlap the switching element TFT. The light-blocking spacer 195 serves as both a spacer for maintaining the thickness of the liquid crystal layer 300 and a black matrix. The light-blocking spacer 195 may be made of a light-blocking material, such as an organic material containing carbon black. The light-blocking material may also be made of a material having a predetermined elasticity. The light-blocking spacer 195 serves as both a black matrix and a spacer for maintaining the thickness of the liquid crystal layer 300. The light-blocking spacer 195, for example, may include a main spacer 195M and a sub spacer 195S. The main spacer 195M is formed to have a height t1 (e.g. thickness) greater than the height t2 of the sub spacer 195S, and may serve to maintain the thickness of the liquid crystal layer 300 even when an external force is applied to the liquid crystal display device 500. The sub spacer 195S may serve to prevent the elasticity of the main spacer 195M from being destroyed by buffering the external force applied to the main spacer 195M when the external force is stronger than the elasticity of the main spacer 195M. The height difference t1-t2 between the main spacer 195M and the sub spacer 195S may be about 0.25 micrometers (μm) to about 0.8 μm. For example, when the main spacer 195M has a height t1 of about 3 μm, the sub spacer 195S may have a height t2 of about 2.5 μm.

The first liquid crystal alignment layer 190 may include an area disposed between the pixel electrode 180 and the liquid crystal layer 300 and an area disposed between the light-blocking spacer 195 and the second liquid crystal alignment layer 270. The first liquid crystal alignment layer 190 may extend to the non-display area II as well as the display area I. The first liquid crystal alignment layer 190 may include a polymer network composed of a polymer of the reactive mesogens, and, for example, may include a polymer network composed of polymers of at least one mesogen of the compound represented by Formula RM1 and the compound represented by Formula RM2.

The polymer network composed of polymers of the reactive mesogens serves to align the liquid crystal compound molecules 301 at a predetermined pretilt angle with respect to the display panel SUB1 and the counter display panel SUB2 even in a state where an electric field is not applied to the liquid crystal display device 500. The pretilt angel means an angle between the display panel SUB1 and the director of the liquid crystal compound molecules 301 and an angle between the counter display panel SUB2 and the director of the liquid crystal compound molecules 301.

Although not shown in the drawings, the first liquid crystal alignment layer 190 may include a polyimide-based alignment base layer and the polymer network composed of polymers of the reactive mesogens, and may further include an alignment stabilizing layer formed on the polyimide-based alignment base layer. However, since the polyimide-based alignment base layer may be omitted, the first liquid crystal alignment layer 190 may not include both the polyimide-based alignment base layer and the alignment stabilizing layer.

The second base substrate 210 is a base substrate of the counter display panel SUB2, and may be made of a transparent insulating material, such as glass or transparent plastic.

The common electrode 250 may be directly disposed on the second base substrate 210. The common electrode 250 may be a pattern electrode having at least one of a protrusion pattern and a slit pattern, or may be a patternless electrode. The common electrode 250 may be made of indium tin oxide, indium zinc oxide, indium oxide, zinc oxide, tin oxide, gallium oxide, titanium oxide, aluminum, silver, platinum, chromium, molybdenum, tantalum, niobium, zinc, magnesium, an alloy thereof, or a laminate thereof.

The second liquid crystal alignment layer 270 may be directly disposed on the common electrode 250. The second liquid crystal alignment layer 250 may include an area disposed between the common electrode 250 and the liquid crystal layer 300 and an area disposed between the common electrode 250 and the first liquid crystal alignment layer 190. The second liquid crystal alignment layer 270 may extend to the non-display area II as well as the display area I. The second liquid crystal alignment layer 270 may include a polymer network composed of polymers of the reactive mesogens, and, for example, may include a polymer network composed of polymers of at least one mesogen of the compound represented by Formula RM1 and the compound represented by Formula RM2.

The polymer network composed of polymers of the reactive mesogens serves to align the liquid crystal compound molecules 301 at a predetermined pretilt angle with respect to the display panel SUB1 and the counter display panel SUB2 even in a state where an electric field is not applied to the liquid crystal display device 500. The pretilt angle means an angle between the display panel SUB1 and the director of the liquid crystal compound molecules 301 and an angle between the counter display panel SUB2 and the director of the liquid crystal compound molecules 301.

Although not shown in the drawings, the second liquid crystal alignment layer 270 includes a polyimide-based alignment base layer and the polymer network composed of polymers of the reactive mesogens, and may further include an alignment stabilizing layer formed on the polyimide-based alignment base layer. However, since the polyimide-based alignment base layer may be omitted, the second liquid crystal alignment layer 270 may not include both the polyimide-based alignment base layer and the alignment stabilizing layer.

Although not shown in the drawing, the liquid crystal display device 500 may further include a backlight assembly (not shown) disposed on the rear surface of the display panel SUB1 to provide light to the liquid crystal layer 300.

The backlight assembly, for example, may include a light guide plate (LGP), a light source, a reflection member, and an optical sheet.

The light guide plate (LGP) serves to direct the path of light emitted from the light source toward the liquid crystal layer 300, and may include a light incidence surface to allow the light emitted from the light source to be applied thereto, and a light emission surface emitting the incident light toward the liquid crystal layer 300. The light guide plate may be made of a material having a predetermined refractive index, such as polymethyl methacrylate (PMMA) or polycarbonate (PC), which is one of light transmissive materials, but is not limited thereto.

When made of such a material, the light incoming onto one side or both sides of the light guide plate has an angle within the critical angle, and the light is transmitted to the inside of the light guide plate. Further, when the incoming light reaches the upper surface or lower surface of the light guide plate, the angle of the light exceeds the critical angle, so that the light is not emitted outside of the light guide plate, and is uniformly transmitted in the light guide plate.

A scattering pattern may be formed on any one of the upper surface and the lower surface of the light guide plate, for example, on the upper surface facing the light emission surface, such that the guided light is emitted to the upper surface thereof. That is, the scattering pattern may be printed on one side of the light guide plate such that the light transmitted in the light guide plate is emitted to the upper surface thereof. Such a scattering pattern may be formed by printing with ink, but is not limited thereto. Further, the light guide plate may be provided with fine grooves or protrusions, and may be variously modified.

The reflection member may further be provided between the light guide plate and the bottom of the storage member. The reflection member serves to reflect the light emitted to the lower surface of the light guide plate, that is, the opposite surface facing the light emission surface and to supply the reflected light to the light guide plate. The reflection member may be fabricated in the form of a film, but is not limited thereto.

The light source may be disposed to face the light incidence surface of the light guide plate. The number of light sources may be appropriately changed as needed. For example, one side of the light guide plate may be provided with one light source, and three or more light sources may also be provided corresponding to three or more sides of the four sides of the light guide plate. Further, a plurality of light source can be provided corresponding to any one of the sides of the light guide plate. While the side light type light source has been described as an example, other examples of the light source may include a direct type light source and a surface shape type light source.

The light source may be a white LED emitting white light, and may also be a plurality of LEDs emitting red light (R), green light (G), and blue light (B), respectively. In the case where a plurality of light sources include the plurality of LEDs emitting red light (R), green light (G), and blue light (B), respectively, when these light sources turn on at once, white light may be produced by color mixing.

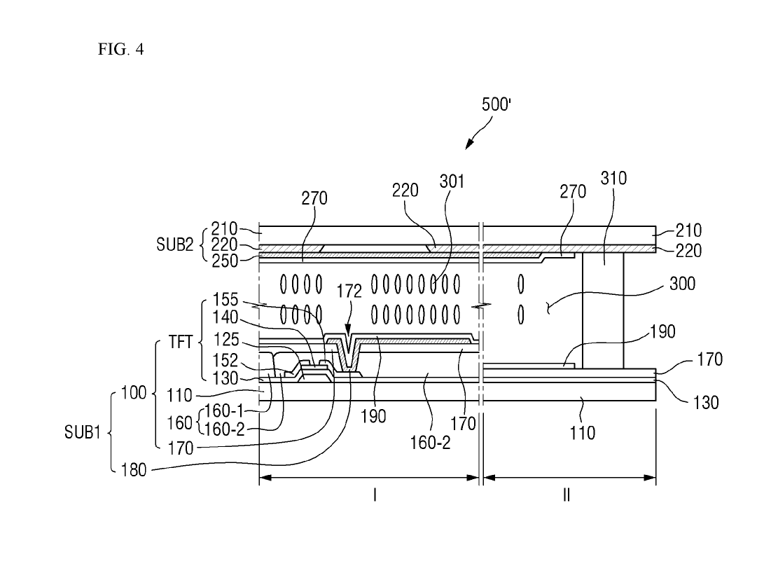

FIG. 4 is a schematic cross-sectional view of the initial state of a second liquid crystal display device 500′, to which an electric field is not applied. Hereinafter, the second liquid crystal display device 500′ will be described with regard to the portions which are different from the first liquid crystal display device (500 of FIG. 2).

The second liquid crystal display device 500′ is different from the first liquid crystal display device 500 of FIG. 2 in that a counter display panel SUB2 is configured to include a second base substrate 210, a light-blocking pattern 220, and a common electrode 250. The counter display panel SUB2 of the first liquid crystal display device 500 of FIG. 2 is configured to include the second base substrate 210 and the common electrode 250. The second liquid crystal display device 500′ does not include the light-blocking spacer 195 shown in FIG. 2. The second liquid crystal display device 500′ may further include a spacer (not shown) for maintaining the thickness of the liquid crystal layer 300, and the spacer may be made of a light transmissive material, and may also include a sealant 310.

As described above, the compound represented by the Formula P has very poor photostability due to a double bond at the end thereof. The voltage holding rate of the second liquid crystal display device 500′ can be improved by partially, or entirely replacing the compound represented by the Formula P with at least one of the compounds represented by Formula 1.

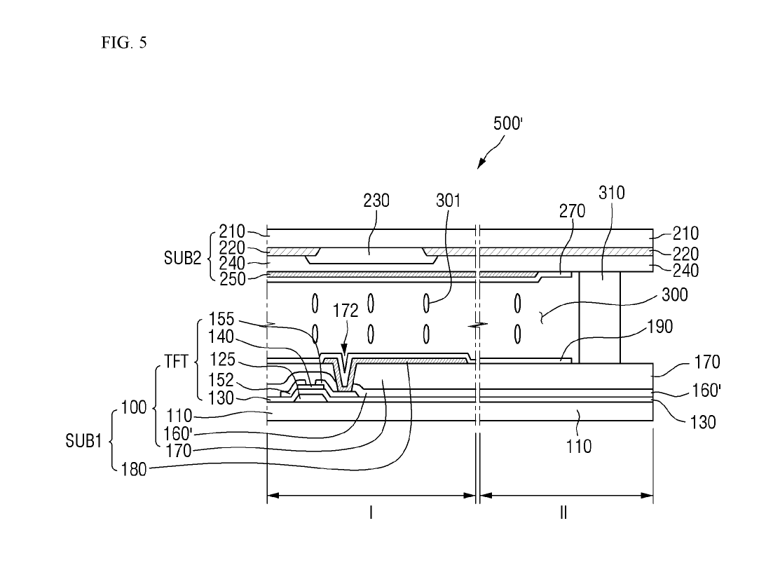

FIG. 5 is a schematic cross-sectional view of the initial state of a third liquid crystal display device 500″, to which an electric field is not applied. Hereinafter, the third liquid crystal display device 500″ will be described with regard to the portions which are different from the first liquid crystal display device 500 of FIG. 2.

The third liquid crystal display device 500″ is different from the first liquid crystal display device 500 of FIG. 2 in that the switching element array substrate 100 is configured to include a first base substrate 110, a switching element TFT disposed on the first base substrate 110, an inorganic film 160′ disposed on the switching element TFT, and an organic film 170 disposed on the inorganic film 160′. The switching element array substrate 100 of the first liquid crystal display device 500 of FIG. 2, for example, is configured to include the first base substrate 110, the switching element TFT disposed on the first base substrate 110, the color filter layer 160 disposed on the switching element TFT, and the organic film 170 disposed on the color filter layer 160.

The third liquid crystal display device 500″ is different from the first liquid crystal display device 500 of FIG. 2 in that a counter display panel SUB2 is configured to include a second base substrate 210, a light-blocking pattern 220, a color filter layer 230, an overcoat film 240, and a common electrode 250. The counter display panel SUB2 of the first liquid crystal display device 500 of FIG. 2 is configured to include the second base substrate 210 and the common electrode 250.

The third liquid crystal display device 500″ does not include the light-blocking spacer 195 shown in FIG. 2. The third liquid crystal display device 500″ may further include a spacer (not shown) for maintaining the thickness of the liquid crystal layer 300, and the spacer may be made of a light transmissive material, and may also include a sealant 310.

As described above, the compound represented by the Formula P has very poor photostability due to a double bond at the end thereof. The voltage holding rate of the third liquid crystal display device 500″ may be improved by partially or entirely replacing the compound represented by Formula P with at least one of the compounds represented by Formula 1.

As described above, according to various embodiments, a low-viscosity liquid crystal composition is provided having high reliability and a liquid crystal display device including the same.

Although the embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A liquid crystal composition, comprising:
at least one of compounds represented by Formula 1: 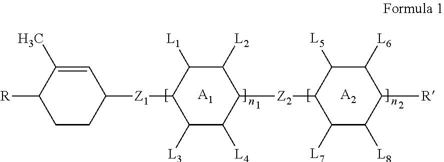
where in the Formula 1, R—* and R′—* are each independently any one of *—H, *—F,*—Cl, *—I, *—Br, a C₁₋₁₂ alkyl group of, a C₁₋₁₂ alkoxy group, or a cyano group; *—Z₁—* and *—Z₂—* are each independently any one of *—C(O)O—*, *—OC(O)—*, *—CF₂O—*, *—OCF₂—*, *—CH₂O—*, *—OCH₂—*, *—SCH₂—*, *—CH₂S—*, *—C₂F₄—*, *—CH₂CF₂—*, *—CF₂CH₂—*, *—(CH₂)ₘ—*, *—CH═CH—*, *—CF═CF—*, *—CH═CF—*, *—CF═CH—*, *—C≡C—*, *—CH═CHCH₂O—*, or a single bond; 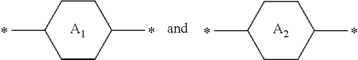
are each independently any one of 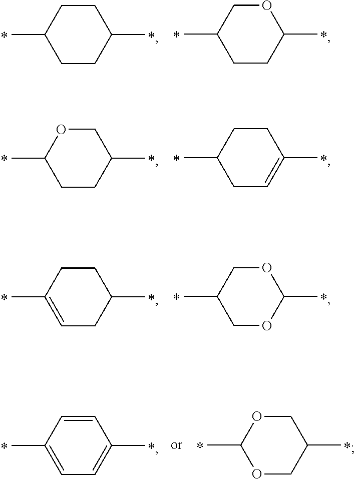
n₁ and n₂ are each independently 0 to 3; m is 2 to 5; and L₁-*, L₂-*, L₃-*, L₄-*, L₅-*, L₆-*, L₇-*, and L₈-* are each independently any one of *—H, *—F, *—Cl, *—OCF₃, *—CF₃, *—CH₂F, or *—CHF₂, where “*” indicates a point of attachment.

2. The liquid crystal composition of claim 1,
wherein at least one of the compounds represented by Formula 1 is any one of compounds represented by Formulae 1-1 to 1-10:

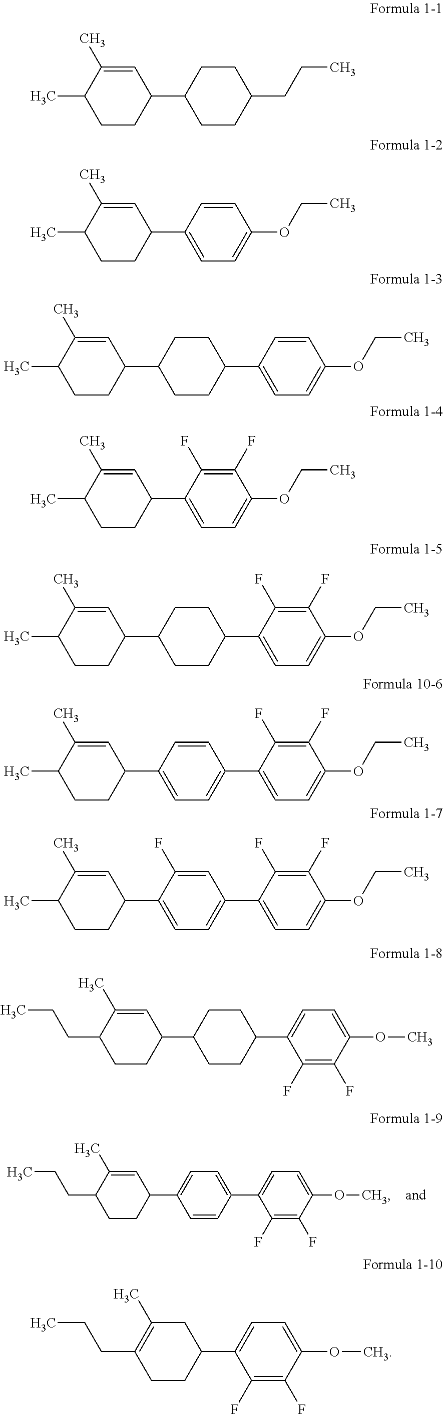

3. The liquid crystal composition of claim 1, further comprising:
at least one of compounds represented by Formulae 2-1 to 2-19: 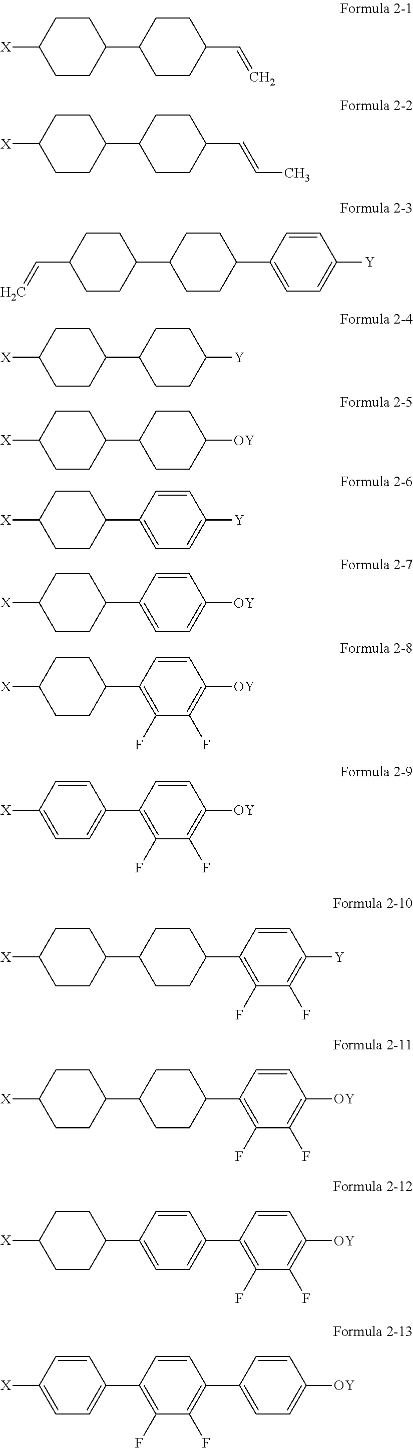 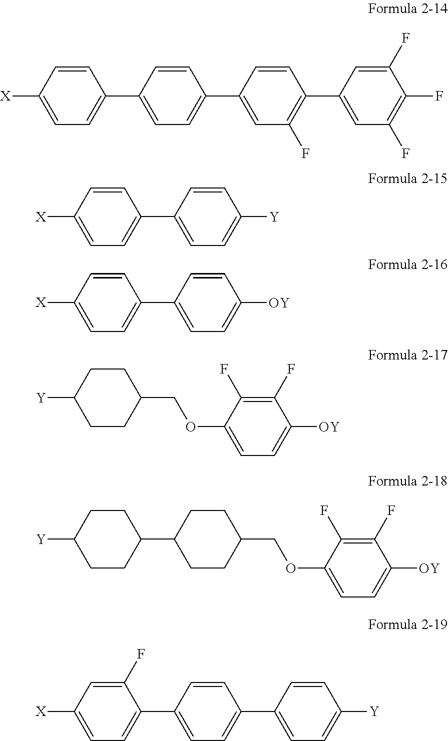
where in the Formulae 2-1 to 2-19, X—* and Y—* are each independently a C₁₋₅ alkyl group.

4. The liquid crystal composition of claim 3,
wherein a content of a compound represented by Formula P below is 0 weight percent:
CH₂═CH—(CH₂)ᵣ-(Cyc)ₛ-(PheF₂)ₜ-R₃₁ Formula P
where in the Formula P, *-Cyc-* is a 1,4-cyclohexylene group; *-PheF₂-* is a 2,3-fluoro-1,4-phenylene group; r is an integer of 0 to 5; each of s and t is an integer of 0 to 3 and a sum of s and t is an integer of 2 to 4; and *—R₃₁ is a C₁₋₅ alkyl group or a C₁₋₅ alkoxy group.

5. The liquid crystal composition of claim 3,
wherein the liquid crystal composition has a refractive index anisotropy of about 0.08 to about 0.12, a dielectric anisotropy of about −2.8 to about −5.5, and a rotational viscosity at 20° C. of about 70 millipascal seconds to about 140 millipascal seconds.

6. A liquid crystal display device, comprising:
a display panel comprising a first base substrate, a switching element disposed on the first base substrate, and a first electrode disposed on the switching element;
a counter display panel comprising a second base substrate and a second electrode disposed on the second base substrate, the counter display panel facing the display panel; and
a liquid crystal layer disposed between the display panel and the counter display panel, wherein the liquid crystal layer comprises at least one of compounds represented by Formula 1 and a reactive mesogen comprising at least one of compounds represented by Formula RM: 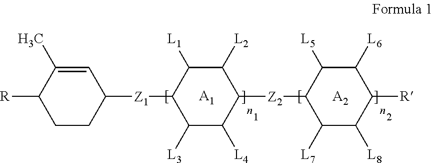
where in the Formula 1, R—* and R′—* are each independently any one of *—H, *—F, *—Cl, *—I, *—Br, a C₁₋₁₂ alkyl group, a C₁₋₁₂ alkoxy group, or a cyano group; *—Z₁—* and *—Z₂—* are each independently any one of *—COO—*, *—OCO—*, *—CF₂O—*, *—OCF₂—*, *—CH₂O—*, *—OCH₂—*, *—SCH₂—*, *—CH₂S—* *—C₂F₄—*, *—CH₂CF₂—*, *—CF₂CH₂—* *—(CH₂)ₘ—*, *—CH═CH—*, *—CF═CF—*, *—CH═CF—*, *—CF═CH—*, *—C≡C—*, *—CH═CHCH₂O—*, or a single bond; 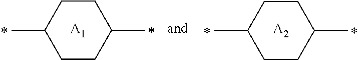
are each independently any one of 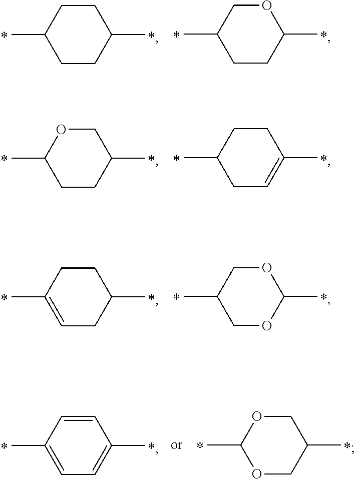
n₁ and n₂ are each independently 0 to 3; m is 2 to 5; and L₁-*, L₂-*, L₃-*, L₄-*, L₅-*, L₆-*, L₇-*, and L₈-* are each independently any one of *—H, *—F, *—Cl, *—OCF₃, *—CF₃, *—CH₂F, or *—CHF₂, where “*” indicates a point of attachment;
P1-SP1-MG-SP2-P2 Formula RM
where in the Formula RM, P1-* and P2-* are each independently any one of 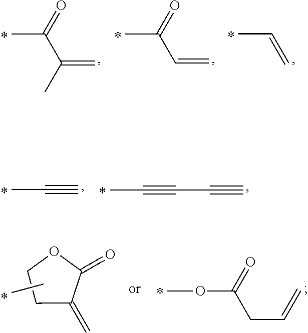
*-SP1-* is *L-Z-L-ArₐL-*, where a is 0 to 2, *-SP2-* is *-LAr-L-Z-L_{b}*, where b is 0 to 2; *-L-* is any one of *—(CH₂)_{c}—*, *—O(CH₂)_{c}—*, 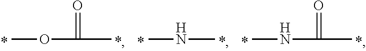
*—CH═CH—*, or *—C≡C—*, *—Z—* is *—(CH₂)_{c}—*, and c is 0 to 12; and *—Ar—* is any one of 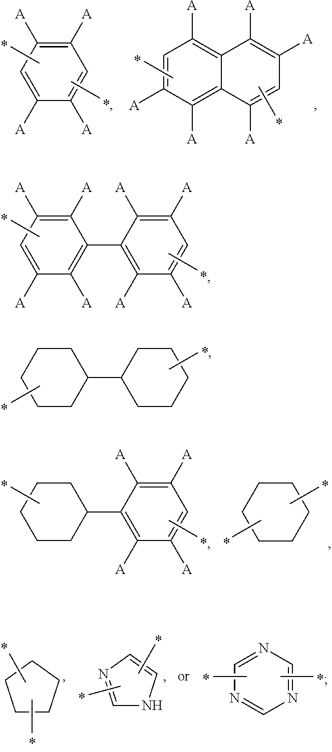
*-MG-* is 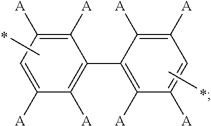
and A-* is any one of H—*, a C₁₋₁₀ alkyl, F—*, Br—*, I—*, *—OH, *—NH₂, or *—CN, where “*” indicates a point of attachment.

7. The liquid crystal display device of claim 6, further comprising:
a liquid crystal alignment layer disposed between the liquid crystal layer and at least one of the display panel and the counter display panel, and comprising a polymer of the reactive mesogen.

8. The liquid crystal display device of claim 6,
wherein at least one of the compounds represented by Formula 1 is any one of compounds represented by Formulae 1-1 to 1-10 below:

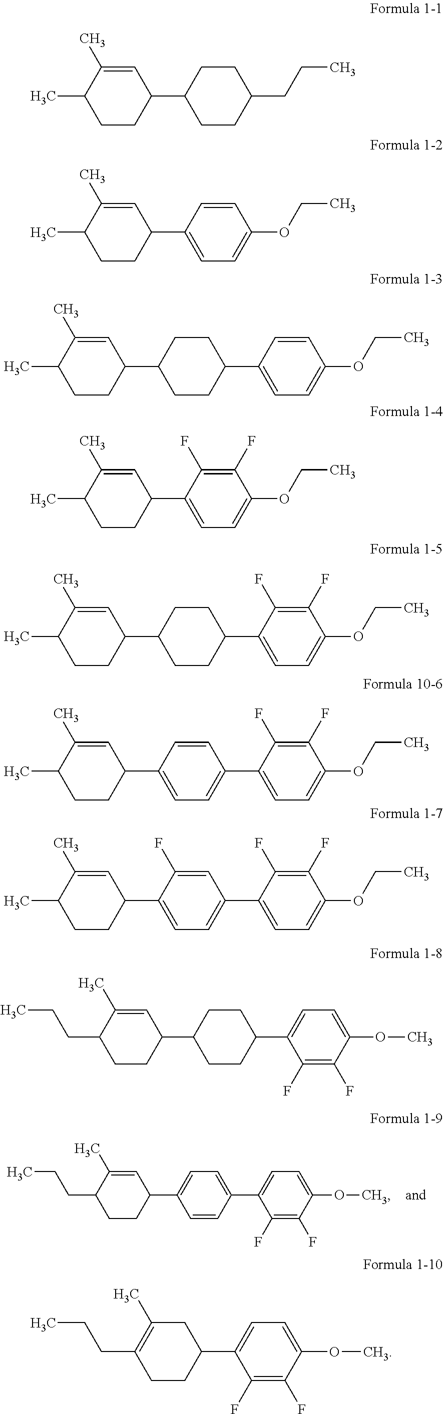

9. The liquid crystal display device of claim 8,
wherein the liquid crystal layer further comprises at least one of compounds represented by Formulae 2-1 to 2-19 below: 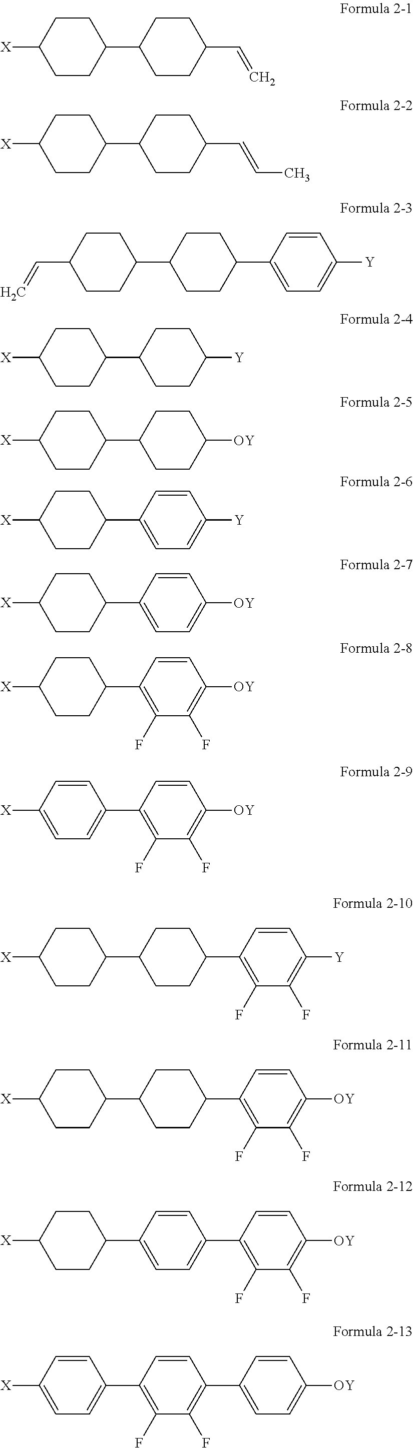 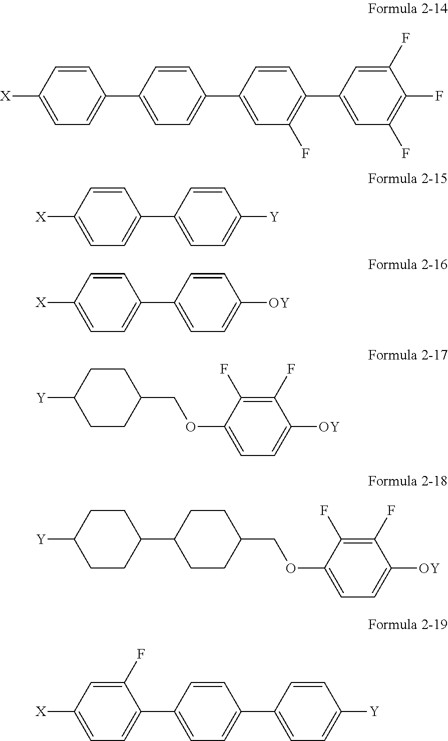
where in the Formulae 2-1 to 2-19, X—* and Y—* are each independently a C₁₋₅ alkyl group.

10. The liquid crystal display device of claim 9,
wherein the content of a compound represented by Formula P below is 0 weight percent
CH₂═CH—(CH₂)ᵣ-(Cyc)ₛ-(PheF₂)ₜ-R₃₁ Formula P
where in the Formula P, *-Cyc-* is a 1,4-cyclohexylene group; *-PheF₂-* is a 2,3-fluoro-1,4-phenylene group; r is an integer of 0 to 5; each of s and t is an integer of 0 to 3 and the sum of s and t is an integer of 2 to 4; and *—R₃₁ is a C₁₋₅ alkyl group or a C₁₋₅ alkoxy group.

11. The liquid crystal display device of claim 7, further comprising:
a light-blocking spacer disposed between the display panel and the liquid crystal alignment layer, wherein the light-blocking spacer comprises a region disposed to overlap the switching element, and wherein a thickness of the liquid crystal layer is determined by a height of the light-blocking spacer.
